(19) United States
(12) Patent Application Publication
(10) Pub. No.: US 2012/0276581 A1
(43) Pub. Date: Nov. 1, 2012

(54) METHOD FOR PRESERVING CANCELLOUS BONE SAMPLES AND PRESERVED CANCELLOUS BONE TISSUE

(75) Inventors: Amir Arav, Tel Aviv (IL); Victor Rzepakovsky, Ness Zionna (IL); Isachar Norman, Gani Tikva (IL); Olga Shneerson, Rehovot (IL); Yehudit Natan, Tel Aviv (IL)

(73) Assignee: CORE DYNAMICS LTD., Hamilton (BM)

(21) Appl. No.: 13/520,721

(22) PCT Filed: Jan. 6, 2011
(86) PCT No.: PCT/IL11/00016

§ 371 Date: Jul. 5, 2012

Related U.S. Application Data
(60) Provisional application No. 61/292,902, filed on Jan. 7, 2010.

(51) Int. Cl. C12N 5/077; C12Q 1/04

(52) U.S. Cl. 435/34; 435/374

(57) ABSTRACT

The claimed subject matter is based on the finding that it is possible to cryopreserve cancellous bone tissue with viable cells (after reconstitution by either thawing or rehydration), the viability after reconstitution being comparable to that of a fresh sample (without freezing) and of beneficial value for use in, for example, transplantation. Thus, provided is a method for cryopreserving a cancellous bone sample. The cryopreserved cancellous bone sample can be in dry form, for example, lyophilized. Also provided is a cryopreserved, in dry form, bone sample and the use of a cryopreserved bone sample; a method for identifying such cryopreserved cancellous bone tissue and uses thereof.

### FIELD OF THE INVENTION

The present invention concerns methods for cryopreserving cancellous bone samples, in particular, drying cancellous bone samples.

### BACKGROUND OF THE INVENTION

Bone allografts are used to fill bone defects caused by trauma, cysts, damages after excision of benign and malignant tumors, joint replacement revisions, congenital defects etc. The purpose of the bone graft is to initiate a healing response of the grafted area and promote new bone formation in the bone graft/native bone interface and the bone graft itself. Biologic bone grafts can be either autologous (autograft) or allograft.

Optimal graft incorporation requires that the bone grafts possess certain qualities. These properties change according to the source of the bone graft. One such quality concerns the bone's osteogenicity. Osteogenicity is the bone graft ability to create new bone, which requires the presence of living bone producing cells. This property can exist in autografts which are immediately transplanted, or bone substitutes enriched with autogenic bone cells culture [Kruyt M C, Dhert W J, Oner C, van Blitterswijk C A, Verbout A J, de Bruijn J D. (2004) Osteogenicity of autologous bone transplants in the goat. Transplantation; 77 (4):504-9.]. Osteoconduction, another required quality, is the bone graft mechanical ability to serve as a scaffold that allows mesenchymal cells to penetrate into it and serve as the matrix in which and the cells can differentiate into bone forming osteogenic cells [Kruyt et al, ibid.]. Osteoinduction referred to the induction of osteogensis by chemicals or proteins. To date, bone grafts (or other treatments) have not demonstrated therapeutic osteogenic capabilities.

Trinity® Multipotential Cellular Bone Matrix (Orthofix, USA) is a viable bone matrix product containing adult stem cells. This product is sold as a frozen product, cryopreserved with 10% DMSO.

Some other orthopedics product of bone tissue do not contain live cells and are sold as frozen/lyophilized sterile products for use as fillers/matrix [“Bone regeneration and repair: biology and clinical applications” edited by Jay R. Lieberman, Gary E. Friedlaender, Humana press 2005; pp:142-143].

A technology for the controlled freezing and thawing of biological samples has been developed and described in International Patent Application Publication No. WO 98/10231. The technology comprises a device for applying laterally varying thermal gradient and a mechanism for moving the sample along the thermal gradient at a controlled velocity rate that provides a variable rate of cooling rates in accordance with a desired protocol.

A method for freeze drying suspended cells using a thermal gradient is described in International Patent Application Publication No. WO 2005/072523. The method provides viable cells post rehydration.

### SUMMARY OF THE INVENTION

The present disclosure is based on the finding that it is possible to cryopreserve, and in particular, dry cancellous bone tissue while maintaining the functionality of the bone cells following reconstitution of the preserved sample. As shown in the following non-limiting examples, the viability after reconstitution was comparable to that of a fresh sample and of significant and beneficial level after storage. The preserved bone sample was found to be useful, for example, for research as well as for transplantation purposes.

Thus, in accordance with one aspect, there is provided by the present disclosure a method for cryopreserving a cancellous bone sample comprising cooling a bone sample comprising cancellous bone tissue in a cryopreservation solution from an initial temperature via an intermediate temperature to a final temperature, the initial temperature being above the freezing point of the cryopreservation solution and the final temperature being below the freezing point, to obtain a frozen cancellous bone sample.

One preferred embodiment of the invention provides an additional step for the method which comprises lyophilizing the frozen sample.

The present invention also provides a cryopreserved, preferably in dry form, bone sample comprising cancellous bone tissue and associated therewith bone cells, at least part of the bone cells being viable post thawing or rehydration.

The cells of the bone tissue are adhered to the tissue and not suspended in the medium surrounding the tissue sample.

Further provided by the present disclosure is the use of a cryopreserved bone sample comprising cancellous bone tissue where at least a portion of bone cells within the cancellous bone tissue are viable after thawing or rehydration, for the preparation of bone graft suitable for transplantation into a subject in need thereof. The invention provides means for identifying the bone sample cells that will be viable post thawing.

Thus, also provided by the invention is a method for identifying and isolating, in needed/desired cancellous bone tissue comprising cells that are viable post rehydration, the method comprising providing a sample comprising cancellous bone tissues and identifying from the sample only cancellous bone tissues that have areas of red-brown, wherein the identified, and if needed, isolated cancellous bone tissue comprises cells that are viable post rehydration.

Also provided by the present disclosure is a kit comprising cryopreserved bone sample comprising cancellous bone tissue wherein at least a portion of the bone cells are viable post thawing or rehydration and instruction for thawing or rehydration of the dried bone sample.

Finally, provided by the present disclosure is a method for providing cancellous bone tissue with viable cells, the method comprising:
providing dry bone sample comprising cancellous bone tissue;
isolating from the dry bone sample, cancellous bone tissue that contains cells viable post thawing;
rehydrating the isolated cancellous bone tissue to provide a rehydrated cancellous bone tissue with viable bone cells.

### DESCRIPTION OF THE FIGURES

In order to understand the invention and to see how it may be carried out in practice, embodiments will now be described, by way of non-limiting example only, with reference to the accompanying drawings, in which:

FIG. 1 is a bar graph showing bone disks and bone chips viabilities before freezing and following thawing or rehydration of samples provided in Table 2.

FIGS. 2A-2B are photographic images taken using an inverted light microscope (Nikon, Japan) of bone disks placed in culture medium; FIG. 2A shows, an image taken after 6 days in culture of freshly harvested bone disks and FIG. 2B is an image taken after 4 days in culture of bone disks that were freeze dried and rehydrated in accordance with the invention.

### DESCRIPTION OF SOME NON-LIMITING EMBODIMENTS

The present invention provides a method for cryopreserving cancellous bone sample comprising cooling a bone sample comprising cancellous bone tissue and a freezing solution from an initial temperature via an intermediate temperature to a final temperature, the initial temperature being above the freezing point of the freezing solution and the final temperature being below the freezing point, to obtain a frozen cancellous bone sample.

In one preferred embodiment, the frozen sample is then placed in a dehydration device, such as a lyophilizer, to obtain a dry cancellous bone sample with cells that are viable after rehydration.

Dehydration of a frozen product allows the providence of a powder dry product. After a material is frozen, a device such as a lyophilizer is used to reduce the surrounding pressure and add enough heat to allow the frozen water in the material to sublime directly from the solid phase to the gas phase, thus providing a dry powder product. The term “dry” is used to denote that the sample comprises not more than 2% water, preferably not more than 1% water and more preferably, no detectable water (detectable by conventional techniques).

The cancellous bone tissue in the context of the disclosure is a sample of bone excised from any part of the endoskeleton of a donor subject and comprising the spongy cancellous bone structure/matrix and bone cells associated (immobilized on/adhered to) with the spongy bone structure. The bone cells include cells, the cells comprise at least one of osteoblasts and mesenchymal stem cells (MSC) but may include others, as detailed below. The donor subject may be a human subject for e.g. autotransplantation or allotransplantation, as well as of xeno donor, such as bovine, porcine and the like, for xenotransplantions.

More specifically, the cancellous bone tissue has a honeycomb structure and consists of blood vessels and different cell types such as adipocytes, hematopoietic stem cells, blood cells, osteoblasts and mesenchymal stem cells typically present in bone tissue. The cells that are of interest when grafting bone chips are the bone forming cells (osteoblasts and mesenchymal stem cells (MSC)) which are known to help repair the damaged bone at the area of grafting. MSC give rise to bone-forming osteoblasts and are thus responsible for bone remodeling and repair. The unique spongy form of the bone tissue rendered the results presented herein unexpected. Specifically, as compared to freezing of cell suspensions where there is partitioning of the suspended (mobile) cells into unfrozen fractions of the sample and thus reduced damage to the cells, it was expected that the immobility of the bone tissue cells (i.e. their being adhered to the bone spongy structure) will result in damage to the cells when cooled below the freezing temperature of the freezing solution, and it was expected that the damage will be to the extent that after reconstitution of the bone sample, either by thawing or by rehydration, the cells will be dead/non-viable. It was even more surprising that the cells survived dehydration following freezing, namely, the lyophiliztion process, and still remain viable.

The cancellous bone tissue may be provided in one or more pieces having the same or various forms, including, without being limited thereto, disks, slices, chips, cylinders, powder, matchsticks or any other desired configuration. In one embodiment, the bone sample excised from a donor subject is sectioned into disk like shapes varying in width and thickness from, without being limited thereto, 0.02 to 2 mm to 0.5 to 30 mm, respectively.

In some embodiments, the bone sample comprises pieces cancellous bone tissue in an average size ranging from 0.2 mm to 1 mm. At times, such sized bone pieces are referred to as bone chips.

Irrespective of its forms, prior to cooling, the cancellous bone pieces are placed in a freezing solution/cryopreservation solution. In some preferred embodiment, the cryopreservation solution is essentially free of permeating cryoprotectants.

Cryoprotectants are agents which are added to a biological sample, such as the bone sample of the invention, in order to minimize the deleterious effects of cryopreservation procedures. Cell injury and death during freezing and thawing of biological cells is related to the formation of large amounts of ice crystals within the cell. Cryopreservation aims to remove intracellular water before freezing so as to reduce the extent of intracellular ice formation to the point where it ceases to constitute a threat to the viability of the cells. Cryoprotectants are thus used to achieve the required intracellular dehydration.

The cryoprotectants may either act by entering the cell and displacing the water molecules out of the cell, such cryoprotectants, are thus known as permeating cryoprotectants; or they act by remaining largely out of the cell but drawing out the intracellular water by osmosis, thus referred to as non permeating cryoprotectants.

The term “permeating cryoprotectants” (also known by the terms “conventional/penetrating/intracellular cryoprotectants”) denotes agents that act by penetrating the cell membrane in the bone sample and reducing the intracellular water concentration, thereby reducing the amount of ice formed at any temperature. Permeating cryoprotectants are typically glycols (alcohols containing at least two hydroxyl groups), such as ethylene glycol, propylene glycol, and glycerol. Examples of permeating cryoprotectants include glycerol, formamide, propanediol, 1-2 propanediol (propylene glycole), dimethylsulfoxide (DMSO), adonitol, methanol, ethylene glycol, dimethyl acetamide, dimethyl formamide. There are risks involved in using cell permeating cryoprotectants, as also described by Gregory M. Fahy et al. with respect to the use of the permeating cryoprotectant dimethyl sulfoxide (DMSO) [Gregory M. Fahy et al. Gryoboiology 27:247-268 (1990)]

The “non permeating cryoprotectants” are agents that a priori do not act by penetrating the cell, but more likely (without being bound thereto) directly on the cell membranes, e.g. involving in changes in colloidal osmotic pressure and modifications of the behavior of membrane associated water by ionic interaction. The non-permeating cryoprotectants are typically polyvinylpyrrolidone, hydroxyethyl starch, monosaccharides, and sugar alcohols.

Examples of non-permeating cryoprotectants include, without being limited thereto, lactose, raffinose, glucose, sucrose, trehalose, D-mannitol, dextrose; proteins such as albumin, cholesterol polyphenol antioxidants such as Epigallocatechin (EGC), Epigallocatechin gallate (EGCG) and antioxidants such as vitamin c, vitamin e, polymers such as polyvinylpyrrolidone (PVP) and carbohydrates such as Dextran, hydroxyethyl starch, cellulose.

In view of the relative toxicity of permeating cryoprotectants, it has been suggested to provide use an alternative cryopreservation solution that is essentially free of permeating cryoprotectants.

The term “essentially free of permeating cryoprotectants” denotes that the solution contains no more than 5% (v/v) permeating cryoprotectant, and at times no more than 2% permeating cryoprotectant or even no more than 1%. According to one embodiment, the cryopreservation solution is free of permeating cryoprotectant. The fact that the cryoprotecting solution is essentially free of a permeating cryoprotectant does not exclude the presence of non-permeating cryoprotectants.

In one embodiment, the cryopreservation solution comprises a combination of non-permeating cryoprotectants. Possible combinations include at least one sugar-based cryoprotectant with any other type of non-permeating cryoprotectant. In one embodiment, the sugar is combined with a low molecular weight cryoprotectant which may be, for example, an anti-oxidant such as EGCG; in some other embodiments, the sugar is combined with a protein, such as albumin.

The bone sample is cooled from a first temperature that is above the freezing point of the cryopreservation solution comprising the cancellous bone tissue to an intermediate that is below the freezing point of the sample.

The term “freezing point of said bone sample” denotes the temperature at which the solution carrying the bone sample, i.e. the cryopreservation solution, starts to freeze. The method is set to operate according to calculated freezing point of the freezing solution. Calculated freezing point of freezing solutions can be easily determined taking into consideration the molality of the solutes in the solvent, and on the type of the solvent (the solvent's cryoscopic constant).

In one embodiment, the cryopreserving solution of the invention is devoid of a permeating cryoprotectant and comprises at least one non-permeating cryoprotectant. The cryopreserving solution typically also comprises an isotonic and non-toxic buffer solution, such as, without being limited thereto, phosphate buffer saline (PBS), saline (0.9% NaCl), DMEM, RPMI-1640 and others which use is acceptable in the field of the invention. This allows the use of a safe cryopreservation solution that does not require the washing of the solution prior treatment and that is solid at room temperature in order to allow for the drying process and ultimately to allow stable storage at >−20° C. temperature. The stable storage refers to storage for at least 24 hours, with no statistically significant reduction in the percentage of viable bone cells post rehydration.

The amount of the non-permeating cryoprotectant may vary depending on the type of the cryoprotectant used, the size and form of the bone sample and the non-toxic buffer solution employed. A person of skill in the art of cryopreservation will be able to select the suitable freezing solution, for use in the method disclosed herein.

In one embodiment, the cryoprotectant comprises Epigallocatechin gallate (EGCG) used in a concentration of between 0.01 mg/ml and 2 mg/ml (about 0.001% and 0.2% (w/v)); in some other embodiments the cryoprotectant comprises trehalose, used in a concentration of between 0.01 and 1.5 M; in yet some other embodiments, the cryoprotectant comprises human serum albumin (HSA) used in a concentration of between 1% (w/v) and 25% (w/v), and any combinations of the above.

The invention also encompasses a cryopreserving solution for cryopreserving, preferably freeze-drying cancellous bone tissue, the solution comprising two or more non-permeating cryoprotectants. In one preferred embodiment, the combination comprises at least one sugar. Possible combinations include EGCG+trehalose or HSA+trehalose as exemplified hereinbelow.

In one embodiment, the sample which is composed of a cancellous bone tissue in a solution essentially free from permeating cryoprotectants is frozen and then placed in a lyophilization system in order to sublimate ice crystals and allow for elevated storage temperatures ≧−20° C., e.g. between −20° C. and room temperature).

Devices that may be employed for changing the temperature of the bone sample in accordance with the invention are described inter alia in U.S. Pat. Nos. 5,873,254, 6,916,602, in US patent application publication No. US-2004-0191754, the content of which is incorporated herein by their entirety by reference. Generally, a device for cooling the bone sample comprises a track; cooling means for imposing a laterally variable temperature gradient along the track; and a mechanism for moving the bone sample along the track. One such device is MTG-1314 freezing apparatus (Core Dynamics, Inc., Nes Tziona, Israel). This freezing apparatus is based on maintaining a thermal gradient in a conductive material and the sample to be frozen is moved at a controlled velocity through this gradient. After seeding is performed at the edge of the sample, ice crystals start to propagate at a velocity which is correlated to the velocity at which the sample passes through a predetermined thermal gradient. Cooling rate, calculated as thermal gradient (G) multiplied by velocity (V), can be precisely controlled.

The method of the invention allows cryopreservation, and in particularly drying, of cancellous bone sample so as to comprise at least 10% of viable cells upon thawing or rehydration of a respectively freeze or freeze dried bone sample. The percent of viable cells in the thawed or rehydrated sample is determined by the percentage of live cells out of the total number of cells in the tested sample. Viability is determined by techniques known in the art, e.g. by staining cells with suitable dyes. Examples of such dyes include, without being limited thereto, Trypan blue, Fluorescein diacetate (FDA), propidium iodide (PI), Syto13, SYBR-14, hochst and other dyes which are acceptable in staining cells.

Preferably, the method of the invention provides a dry cancellous bone tissue, more particularly, lyophilized cancellous bone tissue with bone cells that are viable upon reconstitution of the powder cancellous bone tissue with a suitable buffer.

Thus, the present invention also provides a cryopreserved bone sample comprising cancellous bone tissue and associated therewith bone cells, at least part of the bone cells being viable post thawing or rehydration. Preferably, the cryopreserved bone tissue is freeze dried, namely, in a dry powder form.

When referring to viable cells, the term “at least part of” is used to denote, at least 10% cells are viable post reconstitution (rehydration or thawing), preferably at least 25%, more preferably at least 45% and even 50% or more. The at least a portion of bone cells comprise at least osteoblasts and/or mesenchymal stem cells.

In some embodiment, the cryopreserved bone sample comprises a non-permeating cryoprotectant and at most 5% (v/v), at times, at most 2% of a permeating cryoprotectant and preferably none at all permeating cryoprotectant. Such cryopreserved bone sample comprises a non-permeating cryoprotectant as defined above.

The cryopreserved and in particularly, dry cancellous bone sample can be stably stored at a temperature above −20° C., preferably between −20° C. and room temperature (between ˜25° C. and ˜35° C.).

Also provided by the invention is the use, for the preparation of bone graft suitable for transplantation into a subject in need thereof, of cryopreserved and preferably of dry bone sample comprising cancellous bone tissue where at least a portion of bone cells within the cancellous bone tissue are viable after reconstitution, i.e. thawing or rehydration.

Yet further, there is provided by the invention a kit comprising cryopreserved bone sample comprising cancellous bone tissue wherein at least a portion of the bone cells are viable after reconstitution (e.g. post thawing or rehydration) and instruction for reconstitution of the dried bone sample. The instructions for reconstitution may include instructions for controlled warming of the frozen sample or controlled rehydration of the dried sample. Some non-limiting ways for thawing or rehydration are provided herein below in the experimental section. When the sample is dried, the kit may also include the medium suitable for reconstitution of the dried sample. The kit may be used for providing preserved bone tissue for transplantation or research.

Also provided herein is a method for identifying and if needed isolating from a dry bone sample comprising various pieces of cancellous bone tissue, only those pieces that contain cells that will be viable post rehydration. It has been found that the dry cancellous bone with cells that will be viable post rehydration, comprise areas of red-brown hue as determined by visual inspection. The red-brownish color are of cells, e.g. bone marrow cells that become viable upon rehydration. It is noted that for transplantation or even for research it is essential the preserved cancellous bone tissue essentially maintain its vasculature and cell composition. As well appreciated, the bone, in addition to the hard tissue, consists of blood vessels and different cell types. The existence of the red-brownish color of the dry bone tissue is indicative that the dry tissue has maintained functional blood vessels and cell constitution.

Another method disclosed herein is one for providing reconstituted cancellous bone tissue with viable cells, the method comprising:
providing dry bone sample comprising cancellous bone tissue,
isolating from the dry bone sample, cancellous bone tissue that contains cells viable post thawing;
rehydrating the isolated cancellous bone tissue to provide a reconstituted cancellous bone tissue with viable bone cells.

The method allows the providence of preserved cancellous bone tissue suitable for transplantation.

### NON-LIMITING EXAMPLES

### Materials and Methods

### Bone Disks/Chips Preparation:

Porcine iliac crest bones from female (6 months, 85 kg weight) were brought to Core Dynamics lab from the Institute for Animal Research (Kibutz Lahav, Israel). The bones were collected immediately after slaughter and were carried in foamed plastic box with ice.

From each iliac crest several cylindrical dowels (25-28 mm long and about 6 mm diameter) were drilled from the cancellous area using a 6 mm drill bit. The dowels were placed in 50 ml plastic test tube containing 30 ml PBS including 10% antibiotics (Penicilline-Streptomycin-Amphotericin B). The tubes were shaken gently for a few seconds and then the dowels were transferred into another 50 ml plastic test tube containing 30 ml PBS (10% antibiotics) and for the second time the dowels were washed by gently shaking the tube.

The dowels were then taken out using the McIlwain Tissue Chopper (Mickle laboratory engineering Co. Ltd, UK) with microtome blade and approximately 0.5-1 mm width disks were sliced.

The disks were put in a Petri dish containing PBS (1% antibiotics) where a third washing was performed.

In some experiments disks were cut into smaller pieces (chips) using a scalpel; about 4-5 pieces were cut from each disks, in some other experiments only disks were used.

### Non-Permeating, Cryopreservation Solutions:

Two non-permeating cryopreservation solutions were used:

IMT-2: solution comprising 0.945 mg/ml EGCG and 0.1M trehalose, dissolved in PBS.

IMT-3: solution comprising 0.3M trehalose and 10% (w/v) human serum albumin (HSA) dissolved in PBS.

### Freezing Method

The cancellous bone samples comprising bone disks or bone chips were put into 16 mm diameter glass test tubes which were open at both ends (and closed using crocks). Between 8-10 disks or an amount of chips cut from 8-10 disks were put in each test tube. The bone samples were covered with the appropriate cryopreservation solution (IMT-2 or IMT-3) at a volume of 1.5 ml.

Freezing was done using MTG-1314 freezing apparatus (Core Dynamics, Israel). The temperatures of the system were set as follows: 1° C. (initial temperature), −10° C. (intermediate temperature) and −70° C. (final temperature). The velocity of the movement of the samples in the cooling chamber was 0.05 mm/sec which is calculated to be at a cooling rate of about 0.9° C./min. After freezing at the final temperature, the bone samples were stored in liquid nitrogen (LN) tanks until either thawed or placed in a lyophilizer (Virtis, USA) for 24 hours.

### Thawing

Thawing was performed by immersing the frozen bone samples into a water bath heated to 37° C. and gently moving the tube back and forth until completely thawed.

### Lyophilization

The frozen bone samples were placed in the Virtis lyophilizer for 24 hours. The lyophilization conditions were: shelf temperature set to −55° C. and pressure set 5 mTorr.

### Rehydration

After 24 hours freeze-dried bone samples were taken out of the lyophilizer and rehydrated using PBS heated to 37° C. Specifically, the bone sample containing tubes were held over a Petri dish and heated PBS was poured on the rim of the test tube so as to rehydrate the bone disks or small bone chips and collecting the washed material into the Petri dish.

Some of the chips were separated after freeze drying by color difference (red color was more characteristic to the bone marrow containing bone samples as opposed to the off-white color of the IMT-2 and IMT-3 dried matter (which do not contain (or contain less) bone marrow). It is also possible to separate the bone disks and bone chips by sedimentation because they are heavier then other components in the sample, i.e. other solution solutes. The separated disks were rehydrated directly with PBS or with double distilled water both heated to 37° C.

### Viability Assay

Viability was estimated by live/dead fluorescent stains of Syto13/PI (Invitrogen, USA) observed under a UV microscope before and after thawing of the frozen samples or rehydration of freeze dried samples.

Further, confocal microscope or inverted microscope images were taken and the images were analyzed using Image software (NIH, USA) and cells viability was calculated according to the following formula:

% Viability={number of live cells/(number of live cells+number of dead cells)}×100

### Culture

Some of the disks were placed in a culture medium in order to evaluate the ability of the cells to migrate out of the bone marrow and proliferate. Specifically, bone chips obtained from 8 disks that were cut into 4-5 pieces (about 200 μm-600 μm in size), were placed in a 60 mm diameter culture Petri dish without any medium to let the bone matter adhere to the surface of the dish. After 30 minutes, 5 ml of basal growth medium was added (MEM-Alfa supplemented with 15% (v/v) fetal calf serum and 1% (v/v) Penicillin/streptomycin). The Petri dishes carrying the bone chips were placed in a humidified incubator at 37° C. with 5% CO₂ (Thermo Scientific, USA). After 3 days in culture, the Petri dishes were observed in a converted microscope in order to see if there is any cell migration and to remove non-adherent bone pieces. Every 3 days, the culture medium was replaced with new medium.

In order to count the cells detachment procedure was performed by adding to the Petri dishes 1 ml trypsin-EDTA (after removing the growth medium) and then placing the dishes for 5-10 minutes in the incubator, until all cells have detached. Then 1 ml of growth medium was added to the detached cells and the suspended cells were collected and centrifuged at 200 g for 10 minutes. The supernatant was discarded and 1 ml growth medium was added and suspended cells were taken for viability (live/dead stain) and cell concentration (Hemocytometer counts) assays.

### Statistics

Statistical analysis was done using the 2 way Anova test (JMP software, USA) P<0.05 is considered statistically different.

### Example 1

In a first experiment the following groups were prepared:
1) Disks frozen with IMT-2 solution
2) Disks frozen with IMT-3 solution
3) Chips frozen with IMT-2 solution
4) Chips frozen with IMT-3 solution

From each group 2 samples were frozen and one was thawed (“freeze thaw” sample) and the other lyhophilized and re-hydrated (“freeze-dry” sample) lyophilized. A total of 8 samples were frozen.

TABLE 1
cell concentration* and viability of bone samples following freezing
and thawing or freeze-drying and rehydration

| | | Bone Disks | | Bone Chips | |
|---|---|---|---|---|---|
| | | Cell | | Cell | |
| | | Concen- | | Concen- | |
| Procedure | Solution | tration | Viability | tration | Viability |
| Freeze | IMT-2 | 50% of fresh | 25%-30% | 50% of fresh | 25%-30% |
| thaw | | | | | |
| Freeze | IMT-3 | Similar to | 40%-50% | Similar to | 40%-50% |
| thaw | | fresh | | fresh | |
| Freeze dry | IMT-2 | 30% of fresh | 10%-20% | 30% of fresh | 1%-20% |
| Freeze dry | IMT-3 | 80% of fresh | 40%-50% | 80% of fresh | 40%-50% |

*The cell concentration was normalized (estimated) relative to the concentration in the fresh sample, prior to mixing with the freezing solution.

The above results show that freezing as well as freeze drying, using directional freezing allows for the providence of viable cells post thawing or rehydration. Particularly important is the finding that it is possible to obtain viable cells after drying (freeze drying) of the bone tissue, which is of important commercial value.

It is noted that the percent viability of the cells was determined based respect to the concentration of cells in the fresh sample. The results thus show that a significant amount of cells (as compared to the percent of cells in the sample) remain viable after reconstitution (either thawing or rehydration). The above results are particularly surprising, as it allowed establishing a dry bone sample with viable cells.

Further, the results above show that bone chips survived the process of freezing in the absence of permeating CPAs irrespective of the technique of cryopreservation (freeze thawing or freeze drying and rehydration). The results also show that IMT-3 solution gave better results in terms of cells viability and cells concentration as estimated by observing the stained disks or chips under the UV microscope.

### Example 2

In a second experiment, bone disks frozen with IMT-3 solution were examined. Some samples were examined after freezing and some samples after lyophilization and storage for 24 hours either at room temperature (RT) or in refrigeration (˜4° C.). Viability was assayed using confocal microscopy as well as inverted microscope and freeze dried samples were place in culture before or after storage in order to evaluate the ability of the cells to migrate and proliferate after freeze drying.

A total of 11 bone disk samples were frozen in test tubes according to the following description:

TABLE 2
sample preparations

| | | Storage | |
|---|---|---|---|
| Tube # | Procedure | (duration/temp) | Assay |
| 1 | Freeze-Thawing | 0 | Viability |
| 2 | Freeze-Drying | 0 | Viability |
| 3 | Freeze-Drying | 0 | Viability |
| 4 | Freeze-Drying | 0 | Viability |
| 5 | Freeze-Drying | 0 | Culture |
| 6 | Freeze-Drying | 0 | Culture |
| 7 | Freeze-Drying | 0 | Culture |
| 8 | Freeze-Drying | 24 hrs/RT | Viability |
| 9 | Freeze-Drying | 24 hrs/RT | Culture |
| 10 | Freeze-Drying | 24 hrs/4° C. | Viability |
| 11 | Freeze-Drying | 24 hrs/4° C. | Culture |

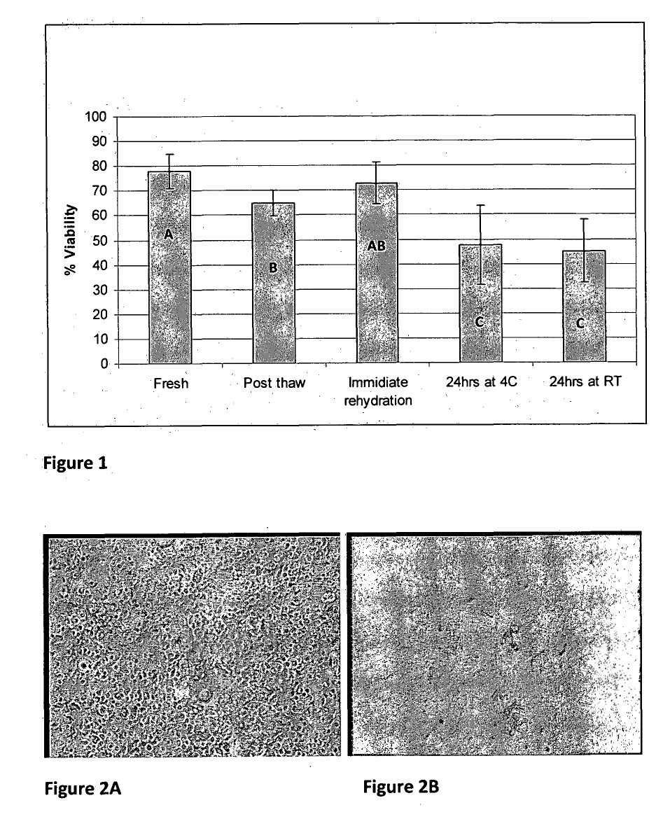

FIG. 1 provides bone disks viabilities before freezing vs. following thawing or rehydration of samples provided in Table 2 (“immediate rehydration” is the average of samples 2 to 4). Immediate rehydration denotes that the sample was not put in storage but was rehydrated as it went out of the lyophilizer. The cells viability was assayed using live/dead fluorescent stains and microscopy observations. Different letters represent statistical difference P<0.05.

The viability results are also summarized in the following Table 3:

TABLE 3
Viability assay

| | | | Rehydration | Rehydration |
|---|---|---|---|---|
| | | Immediate | After | After |
| Fresh | Post Thaw | Rehydration | 4° C. storage | RT storage |
| 77.95% ± 7% | 65% ± | 72.89% ± | 47.6% ± | 45.2% ± |
| | 5.1% | 8.3%* | 16% | 12.7% |

*average of sample 2-4

The results show that at least 45% cells are viable after freeze drying and storage.

With respect to culturing, bone disks that were freeze dried and stored for 24 hours at RT or 4° C. were then rehydrated and placed in culture. After 1 week in culture the cells were assayed for viability and concentration. The bone disks that were stored at RT had a viability of 95.66% (indicative of proliferation) and a concentration of 3.1·10⁶ cells/ml, the bone disks that were stored at 4° C. had a viability of 97.65% (indicative of proliferation) and a concentration of 5.83·10⁶ cells/ml.

The above results show that cancellous bone disks can be lyophilized and stored even at room temperatures (RT). The surviving cells are able to migrate out of the bone marrow and proliferate (the high live/dead ratio) in culture. Namely, the surviving stem cells can propagate.

Furthermore, the viability after lyophilization and immediate rehydration is similar to that of fresh disks. Further, after freeze thawing viability was lower than after immediate rehydration, indicating that the freeze drying technology results in minimal damage to the cells.

The relatively lower viability obtained after storage may be attributed to exposure of the sample to humidity which may have damaged the cells. This is particularly evident in view of the fact that immediate rehydration provided high viability.

### Example 3

In a third experiment, the same conditions as in the second one took place, only with storage for 4 days at refrigeration (4° C.). Furthermore, each disk sample was assayed both for viability and for culture.

The bone disk samples were processed as follows in Table 4.

TABLE 4
Sample preparation:

| Tube # | Procedure | Storage | Assay |
|---|---|---|---|
| 1 | Thawing | No storage | Viability & culture |
| 2 | Freeze-drying | No storage | Viability & culture |
| 3 | Freeze-drying | No storage | Viability & culture |
| 4 | Freeze-drying | 4 days at 4° C. | Viability & culture |

The cell viability of the different samples is provided in Table 5. Viability was determined by live/dead stains and microscope observation.

TABLE 5
Viability Assay

| | | | | After |
|---|---|---|---|---|
| | | | | 4 days |
| | Prior | | Immediate | storage |
| Fresh | Freezing | Post Thaw | Rehydration* | at 4° C. |
| 86.92% ± 7.7% | 86.8% ± 2.6% | 83.8% ± | 67.09% ± | >50%** |
| | | 2.8% | 14.2% | |

*the value is an average of samples 2 and 3
**this sample could not be observed using confocal microscope or regular UV microscope therefore the viability was only estimated by morphological observation.

With respect to cell culture, bone disks prior to freezing (either without the freezing solution (“fresh”) or with the freezing solution (“prior freezing”)), or after freeze drying and storage for 4 days at 4° C. were cultured. The fresh cells were cultured for 6 days while the freeze dried cells were rehydrated and cultured for 4 days. After culturing (4 or 6 days as indicated in the microscope images of FIGS. 2A and 2B) there were adherent cells seen in the Petri dishes indicating that the cells migrated out of the bone disks, adhered onto the Petri dish and proliferated. The cell cultures were imaged and the results are presented in FIGS. 2A and 2B. The same findings were exhibited using confocal microscope (data not shown).

The above results show that the bone disks not only survive after freeze drying and immediate rehydration, but also after 4 days in a refrigerator where they maintained high viability and furthermore their ability to grow in culture.

### Example 4

In the following experiment very small bone chips (50 μm) were frozen and thawed. In order to evaluate the survival of mesenchymal stem cells (MSC) in which are in the cancellous bone the cells were extracted out of the bone marrow in the bone chips using collagenase.

Specifically, bone disks were prepared as described above, and then the disks were further cut into small chips of 50 μm in size using a scalpel. Two samples containing small chips were re-suspended with IMT-3 cryopreservation solution, frozen using the MTG-1314 and thawed as described above.

The collagenase procedure (performing collagen breakdown) was performed on the samples both prior to freezing (after being mixed with IMT-3 solution) and after freezing and thawing. The collagenase procedure was done as follows: the bone chips were incubated for 10 minutes at room temperature with 0.12% trypsin-EDTA (Biological Industries Ltd, Beit Haemek, Israel) solution in PBS (Biological Industries Ltd, Beit Haemek, Israel). Then, the bone chips were transferred into 50 ml plastic test tubes that contained 3 ml of 0.3% collagenase (Sigma Aldrich, St. Louis, USA) in DMEM (Biological Industries Ltd, Beit Haemek, Israel). The test tubes were then incubated for 30 minutes in a water bath warmed to 37° C. during which the samples were shaken. After 30 minutes the supernatant was discarded and to the remaining bone chips 3 ml of fresh collagenase solution was added and samples were incubated again for 30 minutes in a water bath heated to 37° C. and shaken during this period. Then the supernatant was collected and centrifuged for 5 minutes at 200 g. Again, the supernatant was discarded and the cells pellet was re-suspended with 7 ml of DMEM (Biological Industries Ltd, Beit Haemek, Israel) and centrifuged again for 5 minutes at 200 g. After this second centrifugation the supernatant was discarded and the cells pellets were re-suspended with 1 ml of DMEM (Biological Industries Ltd, Beit Haemek, Israel) and a sample of 100 μl was taken for viability and cells counts assays.

The viability of the cells following the above procedure is summarized in Table 6. It is noted that the two samples contained bone chips taken from the same animal.

TABLE 6
Cell viability

| | % Viability | Average % Viability |
|---|---|---|
| Prior Freezing #1 | 49.24% | 59.5 ± 9.36 |
| Prior Freezing #2 | 70.08% | |
| Post Thaw #1 | 29.3 ± 6.3 | 35.5 ± 5.92 |
| Post Thaw #2 | 41.7 ± 5.53 | |

The above results show that the MSC survive the freeze-thawing procedure. Since the MSC are embedded within the bone marrow only treatment with collagenase can remove them out of the bone matrix. Although there are also osteoblasts and other cell types, all of them are bone forming cells and not hematopoietic stem cells which are non-adherent cells and are washed out during the collagenase procedure.

Further, when comparing the post thaw results to those obtained prior freezing 49.24% and 70.08% of the cells have survived the freeze thawing process.

1.-23. (canceled)

24. A method for cryopreserving a cancellous bone sample comprising (a) cooling a bone sample comprising cancellous bone tissue in a cryopreservation solution from an initial temperature via an intermediate temperature to a final temperature, the initial temperature being above the freezing point of the cryopreservation solution and the final temperature being below the freezing point, to obtain a frozen cancellous bone sample, and (b) lyophilizing the frozen cancellous bone sample to obtain a lyophilized cryopreserved cancellous bone tissue.

25. The method of claim 24, wherein the bone tissue is in the form of bone pieces of the same or different width and thickness, the width being between 0.02 to 2 mm to and the thickness between 0.5 to 30 mm.

26. The method of claim 24, wherein the cryopreservation solution comprises a non-permeating cryoprotectant.

27. The method of claim 24, wherein the cryopreservation solution comprises at most 2% permeating cryoprotectant.

28. The method of claim 24, wherein the cryopreservation solution is essentially free of permeating cryoprotectant.

29. The method of claim 24, wherein the initial temperature is in the range of from 0° C. to 25° C., the intermediate temperature is in the range of from −1° C. to −100° C. and the final temperature is in the range of from −20° C. to −100° C.

30. The method of claim 24, comprising storing the dried bone sample at a temperature between −20° C. and 35° C., for a period of at least 24 hours.

31. The method of claim 24, wherein the bone sample comprises a combination of non-permeating cryoprotectants.

32. The method of claim 31, wherein the bone sample comprises a combination of two or more non-permeating cryoprotectants selected from the group consisting of lactose, raffinose, sucrose, trehalose, D-mannitol, dextrose; human serum albumin, bovine serum albumin, cholesterol, polyphenol, epigallocatechin (EGG), Epigallocatechin gallate (EGCG), vitamin C, vitamin E, polyvinylpyrrolidone (PVP), Dextran, hydroxyethyl starch, and cellulose.

33. A cryopreserved bone sample comprising lyophilized cancellous bone tissue and associated therewith bone cells, at least part of the bone cells being viable post rehydration.

34. The cryopreserved bone sample of claim 33, comprising a non-permeating cryoprotectant and at most 2% (v/v) of a permeating cryoprotectant.

35. The cryopreserved bone sample of claim 33, essentially free of permeating cryoprotectant.

36. The cryopreserved bone sample of claim 31, comprising a combination of two or more non-permeating cryoprotectants.

37. The cryopreserved bone sample of claim 36, comprising a combination of two or more non-permeating cryoprotectants.

38. The cryopreserved bone sample of claim 34, comprising a non-permeating cryoprotectant selected from the group consisting of lactose, raffinose, sucrose, trehalose, D-mannitol, dextrose; human serum albumin, cholesterol polyphenol, epigallocatechin (EGO), Epigallocatechin gallate (EGCG), vitamin C, vitamin E, polyvinylpyrrolidone (PVP), Dextran, hydroxyethyl starch, and cellulose.

39. The cryopreserved bone sample of claim 33, being stably stored at a temperature between −20° C. and 35° C.

40. The cryopreserved bone sample of claim 33, comprising, post rehydration, at least 10% bone cells selected from osteoblasts and mesenchymal stem cells.

41. A method for preparing a bone graft suitable for transplantation into a subject in need thereof, comprising: providing a cryopreserved bone sample comprising lyophilized cancellous bone tissue where at least a portion of bone cells within the cancellous bone tissue are viable after rehydration.

42. A kit comprising cryopreserved bone sample comprising lyophilized cancellous bone tissue wherein at least a portion of the bone cells are viable post rehydration and instructions for rehydration of the dried bone sample.

43. The kit of claim 42, for use in transplantation.

44. A method for identifying cancellous bone tissue comprising cells that are viable post rehydration, the method comprising providing a sample comprising cancellous bone tissues and detecting from the sample only cancellous bone tissues that have areas of red-brown, wherein the isolated cancellous bone tissue comprises cells that are viable post rehydration.

45. A method for providing cancellous bone tissue with viable cells, the method comprising:
a. providing dry bone sample comprising lyophilized cancellous bone tissue,
b. identifying in the dry bone sample, cancellous bone tissue that contains cells viable post rehydration; and
c. rehydrating the identified cancellous bone tissue to provide a rehydrated cancellous bone tissue with viable bone cells.
